(19) United States
(12) Patent Application Publication
(10) Pub. No.: US 2025/0022544 A1
(43) Pub. Date: Jan. 16, 2025

(54) INTERACTION IMPACT EVALUATION METHOD, ELEMENT MATERIAL IDENTIFYING METHOD, AND SUBSTITUTE MATERIAL SEARCH METHOD

(71) Applicant: Konica Minolta, Inc., Tokyo (JP)

(72) Inventors: Yukihito NAKAZAWA, Tokyo (JP); Michihiro OKUYAMA, Tokyo (JP); Tomohiro OSHIYAMA, Tokyo (JP)

(21) Appl. No.: 18/714,949

(22) PCT Filed: Nov. 9, 2022
(86) PCT No.: PCT/JP2022/041710

§ 371 Date: May 30, 2024

(30) Foreign Application Priority Data
Dec. 10, 2021 (JP) 2021-201146

(51) Int. Cl. G16C 20/30; G16C 20/40; G16C 20/70

(52) U.S. Cl. CPC G16C 20/30; G16C 20/40; G16C 20/70

(57) ABSTRACT

An interaction impact evaluation method that enable highly accurate prediction of properties or a search for new substitute materials having desired properties is provided. According to the present invention, a step for selecting a kind of interaction due to a plurality of element materials, and a step for evaluating the degree to which the selected interaction is involved with the property of the composite material are performed to evaluate the impact of interaction with respect to the property of a composite material including a plurality of types of element materials.

### TECHNICAL FIELD

The present invention relates to a method of evaluating influence of interaction, a method of specifying an element material, and method of searching for an alternative material.

### BACKGROUND ART

Composite materials to which a plurality of types of materials are added are used for various applications in order to obtain characteristics such as high mechanical strength and high heat resistance. In these composite materials, changes in characteristics due to each material (hereinafter, each material constituting the composite material is also referred to as “element material”) often do not occur as expected. Therefore, there are circumstances where it is difficult to determine how element materials should be combined in order to obtain desired characteristics. In practice. the determination of the combination of element materials is often left to the intuition of an experienced craftsman.

In consideration of such circumstances, development of a method of predicting characteristics of a composite material has been advanced. For example, Patent Literature 1 describes a physical property information estimation method in which a plurality of pieces of formulation information about an adhesive are input to a learned support vector machine to estimate physical property information corresponding to each piece of formulation information, and physical property information satisfying a predetermined criterion and formulation information corresponding thereto among the obtained physical property information are output.

### CITATION LIST

### Patent Literatures

### PTL 1

Japanese Unexamined Patent Publication No. 2021-26478

### SUMMARY OF INVENTION

### Technical Problem

As described in PTL 1, a method has been studied in which a prediction model is generated by machine learning using a known formulation and its characteristics as teacher data, and the prediction model is used to predict characteristics to be obtained by a new formulation.

However, according to the findings of the present inventors, with the prediction models generated by these methods, although the prediction accuracy could be increased within the ranges of the materials and the blending amounts thereof included in the teacher data, the prediction accuracy of the characteristics was not increased as expected with respect to the blending using the materials not included in the teacher data or the like in some cases.

In addition, for the purpose of stabilization of material supply, cost, adjustment of other physical properties, reduction of influence on the environment, and the like, an element material constituting an existing composite material may be changed to another material. Even in such a case, it is desired to predict, with higher accuracy than before, which element material to be replaced among the element materials in the composite material would be less likely to change the characteristics of the composite material (or more likely to change the characteristics of the composite material). In addition, even when an element material to be replaced has been determined, it is desired to predict with high accuracy which material among candidates for a new material for replacement would obtain the same level of characteristics as the original or more improved characteristics.

The present invention has been made in view of the above circumstances, and it is an object of the present invention to provide a method of evaluating influence of interaction, a method of specifying an element material, and a method of searching for an alternative material that make it possible to predict characteristics with high accuracy or search for a new alternative material having desired characteristics

### Solution to Problem

The problem is solved by a method of evaluating influence of interaction, the method including: selecting, for a composite material including a plurality of types of element materials, a type of interaction by two or more element materials of the plurality of types of element materials; and evaluating a degree of involvement of the selected interaction to a characteristic possessed by the composite material.

In addition, the above-described problem is solved by a method of specifying an element material, the method including: performing the method of evaluating influence of interaction; and specifying, based on the degree of involvement obtained by the method of evaluating influence of interaction, the element material corresponding to a degree to which the characteristic changes when the element material is replaced.

Furthermore, the above-described problem is solved by a method of searching for an alternative material that replaces an element material of a composite material including a plurality of types of element materials, the element material being among the plurality of types of element materials, the method including; specifying the element material to be replaced among the plurality of types of element materials by the method of specifying an element material: acquiring a feature amount of the element material to be replaced; and extracting an alternative material from a candidate for the alternative material based on the acquired feature amount.

### Advantageous Effects of Invention

According to the present invention, provided are a method of evaluating influence of interaction, a method of specifying an element material, and a method of searching for an alternative material that enable highly accurate prediction of characteristic or enable search for a new alternative material having desired characteristic.

### BRIEF DESCRIPTION OF DRAWINGS

FIG. 1 is a flowchart illustrating a method of evaluating influence of interaction according to an embodiment of the present invention;

FIG. 2 is a flowchart illustrating a method of performing a step S130 in FIG. 1:

FIG. 3 is a flowchart illustrating a method of specifying an element material according to another embodiment of the present invention:

FIG. 4 is a flowchart illustrating a method of searching for an alternative material according to another embodiment of the present invention;

FIG. 5 is a histogram of the flexural modulus of 38 types of composite materials, in which the horizontal axis represents the flexural modulus (GPa) and the vertical axis represents the frequency, in the examples,

FIG. 6 is a graph illustrating the absolute value of the residual according to each of expression (1) and expression (2) for each of six test data in the example;

FIG. 7A is a graph illustrating Rn according to the expression (18) and Ri according to the expression (19) obtained from the test data D in the examples, and FIG. 7B is a graph illustrating Rᵢ₂ and Rᵢ₃ according to the expression (20) obtained from the test data D in the examples, and FIG. 7C is a graph illustrating R_{mf}, Rₘₐ and R_{fa} according to the expression (21) obtained from the test data D in the examples; and

FIG. 8 illustrates an execution result of ISOMAP in the examples.

### DESCRIPTION OF EMBODIMENTS

Hereinafter, embodiments of the present invention will be described in detail with reference to the drawings. Note that the present invention is not limited to the following forms.

The present inventors have considered that a main effect, in which each element material independently changes characteristics, and interaction due to a combination of a plurality of types of element materials are involved in characteristics of a composite material (for example, characteristics of a composite material which change depending on a combination of element materials, such as flexural modulus and heat resistance). Then, the present inventors have considered that in order to accurately predict a change in characteristics when an element material is changed from that of a known composite material, it is necessary to incorporate the contribution of interaction into the prediction. For example, since the prediction model used in the method described in PTL 1 cannot be said to sufficiently reflect the influence of interaction, it is considered that prediction accuracy does not sufficiently increase when a new material that is not included in teacher data is applied to a composite material (i.e., when a new interaction occurs).

Also, when considering new composite materials, one or more of types of the element materials of known composite materials are often replaced with new materials. In such a case, it is considered that the influence of the interaction on the characteristics of the known composite material is evaluated, and the alternative material is determined and changed based on the magnitude of the influence of the interaction, whereby the characteristics of the composite material after the replacement can be predicted with higher accuracy.

### Method of Evaluating Influence of Interaction

One embodiment of the present invention based on the above-described new finding relates to a method of evaluating influence of interaction on characteristics of a composite material

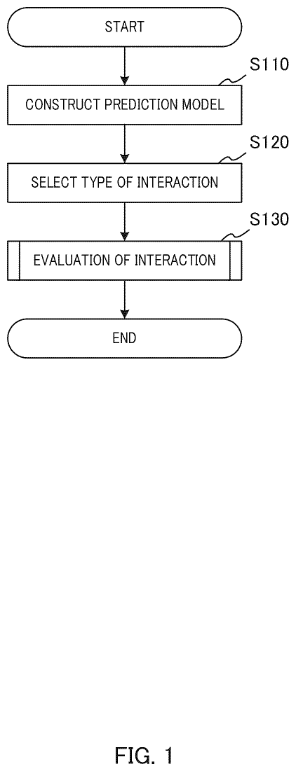

FIG. 1 is a flowchart illustrating an evaluation method according to the present embodiment. The evaluation method according to the present embodiment includes: a step of constructing a prediction model capable of predicting a characteristic possessed by a known composite material (step S110); a step of selecting a type of interaction to be evaluated among interactions included in the composite material (step S120); and a step of evaluating the degree of involvement of the selected interaction to the characteristic possessed by the composite material (step S130). Note that in the present embodiment, it is not necessary to perform all of these steps, and for example, step S110 may be omitted when a prediction model has already been constructed.

Step S110 is a step of constructing a prediction model capable of predicting characteristics of a known composite material.

The prediction model may be constructed using a plurality of composite materials whose compositions and characteristics are known, including the above known composite material, as teacher data by a known method such as partial least squares regression (PLS), a neural network, a decision tree, support vector regression, principal component regression (PCR), ridge regression, kernel based PLS, or Gaussian step regression (GPR).

As the prediction model, for example, a model using a linear prediction expression (1) below is known.

$$y = {\sum }_{i = 1}^{{n}_{x}} {c}_{i} {x}_{i} + {c}_{0} \qquad [ Expression 1 ]$$

In expression (1), y is an objective variable, c₀ is a constant term, c_{I} is an i-th partial regression coefficient, x_{I} is an i-th explanatory variable, and nₓ is the number of explanatory variables. In the present embodiment, y may be a certain characteristic possessed by the composite material, nₓ may be the number of element materials, and X₁ to Xₙₓ may be the addition rates of the first to nₓth element materials.

Step S120 is a step of selecting interaction whose degree of involvement is to be evaluated.

There are a wide variety of interactions in a composite material. For example, in a composite material including three types of element materials of an element material A, an element material B, and an element material C, there are interaction between the element materials A and B. interaction between the element materials A and C, interaction between the element materials B and C, and interaction between the element materials A, B, and C. In this step, a type of interaction meeting a criterion may be selected from among these interactions. Selecting the “type” of interaction means that only a single interaction may be selected, or a group of a plurality of interactions belonging to the same series (for example, the number of element materials constituting the interaction is the same) may be selected.

For example, when it is desired to evaluate the degree of involvement of interaction between element materials A and B to the characteristics of a composite material containing the above-described three element materials, only “interaction between element materials A and B” may be selected. Alternatively, when it is desired to evaluate the degree of involvement of the interaction between two types of element materials to the characteristics, “all of the interaction between the element materials A and B, the interaction between the element materials A and C, and the interaction between the element materials B and C” may be selected, and when it is desired to evaluate the the degree of involvement of the interaction between three types of element materials to the characteristics, “the interaction between the element materials A, B, and C” may be selected. In addition, in a case where it is desired to evaluate the degree of involvement of the entirety of the interaction in the characteristics of the composite material, “all of the interaction between the element materials A and B. the interaction between the element materials A and C, the interaction between the element materials B and C, and the interaction between the element materials A, B, and C” may be selected. Alternatively, without specifying a specific interaction, only whether or not the degree of involvement of the interaction in the characteristics of the composite material is large may be selected. Thus, according to the selection of the interaction to be evaluated, a single interaction or a combination of a plurality of interactions is appropriately specified in this step.

Step S130 is a step of evaluating the degree of involvement of the interaction specified in the previous step to the characteristics of the composite material.

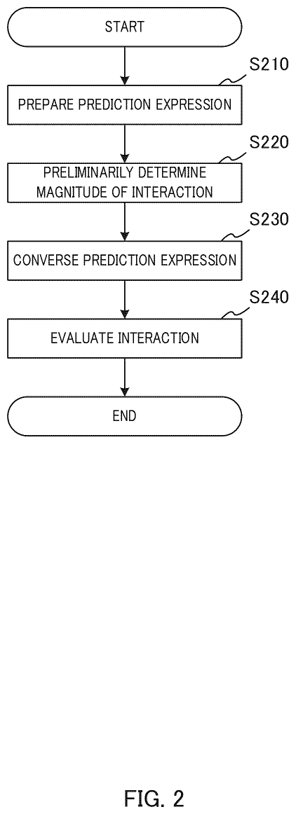

FIG. 2 is a flowchart illustrating a method of performing step S130 in the present embodiment. The step S130 includes a step (step S210) of preparing a nonlinear prediction expression, a step (step S220) of preliminarily determining the degree of the magnitude of the interaction, a step (step S230) of converting the prediction expression prepared in the step S210 into a form in which an output value indicating the contribution of the linear term or the contribution of the interaction can be separated and output, and a step (step S240) of evaluating the degree of involvement of the specified interaction in the characteristics of the composite material. Note that in the present embodiment, it is not necessary to perform all of these steps, and for example, step S220 may be omitted.

In a step S210, a nonlinear prediction expression for predicting a characteristic Q for evaluating influence of interaction is prepared for a composite material α.

According to the finding of the present inventors, in the prediction expression, the interaction is represented by a nonlinear term defined as the product of the feature amounts of a plurality of element materials (for example, xᵢⁿⁱxⱼ^{nj} for the interaction by two types of element materials I and j). Therefore, in order to evaluate the degree of involvement of the interaction in the characteristics of the composite material, a nonlinear prediction expression is preferably used.

In the present embodiment, the following nonlinear prediction expression (2) is used in the following steps.

$$ln y = {\sum }_{i = 1}^{{n}_{x}} {c}_{i} {x}_{i} + {c}_{0} \qquad [ Expression 2 ]$$

In expression (2), y is an objective variable, c₀ is a constant term, c_{I} is an i-th partial regression coefficient, x_{I} is an i-th explanatory variable, and nₓ is the number of explanatory variables. In the present embodiment, y may be a certain characteristic Q possessed by the composite material, nₓ may be the number of element materials, and x₁ to Xₙₓ may be the addition rates of the first to nₓth element materials.

In Step S220, the degree of the magnitude of the interaction related to the characteristic Q of the composite material α is preliminarily determined from expression (1) and expression (2).

The interaction is expressed by a nonlinear term, and the magnitude of the nonlinear term in expression (2) increases as the degree of involvement of the interaction increases. Therefore, by determining the magnitude of the nonlinear term in expression (2), the degree of the magnitude of the interaction can be preliminarily determined.

Specifically, the absolute value of the residual between the value of the characteristic Q that the composite material α actually has and the predicted value of the characteristic Q predicted from expression (1) is obtained, and this is set as ε₍₁₎. In addition, the residual between the value of the characteristic Q that the composite material α actually has and the predicted value of the characteristic Q predicted from expression (2) is obtained, and is set as ε₍₂₎.

Then, ε₍₁₎ and ε₍₂₎ are compared, and when ε₍₂₎ is smaller than ε₍₁₎, it can be said that the expression (2) including the nonlinear term can predict the characteristic Q of the composite material α more accurately than the expression (1) not including the nonlinear term. In addition, since the magnitude of the nonlinear term in expression (2) is large for the characteristic Q that the composite material α actually has, it can be said that the accuracy of prediction was low with expression (1) that does not include a nonlinear term. Since it can be said that the degree of involvement of the interaction is larger as the nonlinear term becomes larger, at this time, it can be preliminarily determined that the degree of involvement of the interaction in the characteristic Q of the composite material α is larger.

On the other hand, when ε₍₁₎ and ε₍₂₎ are about the same, or when ε₍₂₎ is larger than ε₍₁₎, it can be preliminarily determined that the nonlinear term is small and the magnitude of the interaction is also small.

When “whether or not the degree of involvement of the interaction in the characteristic Q of the composite material α is large” is selected in the step S120, the preliminary determination result in the step S220 may be used as the determination result in the step S130.

In the step S230, the nonlinear prediction expression prepared in the step S210 is converted into a format in which an output value indicating a contribution of the linear term can be separated from an output value indicating other contributions and output, and a format in which an output value indicating a contribution of each interaction can be separated from an output value indicating other contributions and output.

In the present embodiment, the composite material α is composed of three components. a matrix resin m, a filler f, and an additive a. At this time, the expression (2) can be expressed by the following expression (3), and the expression (3) can be converted into the expression (4) as an exponential function indicating the characteristic y_{α} of the composite material α.

$$\begin{matrix}ln {y}_{α} + {c}_{{α}_{m}} {x}_{{α}_{m}} + {c}_{{α}_{f}} {x}_{{α}_{f}} + {c}_{{α}_{a}} {x}_{{α}_{a}} + {c}_{0} & [ Expression 3 ] \\ {y}_{α} = {Ae}^{{c}_{{α}_{m}} {x}_{{α}_{m}}} {e}^{{c}_{{α}_{f} {x}_{{α}_{f}}}} {e}^{{c}_{{α}_{a}} {x}_{{α}_{a}}} & [ Expression 4 ]\end{matrix}$$

In expression (3) and expression (4), C_{αm} and C_{αf}, and C_{αa} are partial regression coefficients of the matrix resin m, the filler f, and the additive a in the composite material α, respectively. In addition, x_{αm}, x_{αf}, and x_{αa} are feature amounts of the matrix resin m, the filler f, and the additive a in the composite material α, respectively, and are addition rates thereof in the present embodiment. Further, y_{α} is a characteristic y of the composite material α, and A is a constant.

Furthermore, when expression (4) is Taylor-expanded around the origin, the following expression (5) is obtained.

$${y}_{α} = {\sum }_{{n}_{m} , {n}_{f} , {n}_{a} = 0}^{∞} ( A \frac{{c}_{{α}_{m}}^{{n}_{m}} {c}_{{α}_{f}}^{{n}_{f}} {c}_{{α}_{a}}^{{n}_{a}}}{{n}_{m} ! {n}_{f} ! {n}_{a} !} ) {x}_{{α}_{m}}^{{n}_{m}} {x}_{{α}_{f}}^{{n}_{f}} {x}_{{α}_{a}}^{{n}_{a}} \qquad [ Expression 5 ]$$

Since expression (5) includes the products of the feature amounts x_{αm}, x_{αf} and x_{αa}, it can be seen that interaction is expressed in this expression.

Expression (5) can be further decomposed into a term represented by one type of feature amount, a term including the product of two types of feature amounts, and a term including the product of three types of feature amounts, and represented by expression (6).

$${y}_{α} = A ( 1 + {\sum }_{{n}_{m} = 1}^{∞} \frac{{c}_{{α}_{m}}^{{n}_{m}}}{{n}_{m} !} {x}_{{α}_{m}}^{{n}_{m}} + {\sum }_{{n}_{f} = 1}^{∞} \frac{{c}_{{α}_{f}}^{{n}_{f}}}{{n}_{f} !} {x}_{{α}_{f}}^{{n}_{f}} + {\sum }_{{n}_{a} = 1}^{∞} \frac{{c}_{{α}_{a}}^{{n}_{a}}}{{n}_{a} !} {x}_{{α}_{a}}^{{n}_{a}} ) + {\sum }_{{n}_{m} , {n}_{f} = 1}^{∞} \frac{{c}_{m {α}_{m}}^{{n}_{m}} {c}_{{α}_{f}}^{{n}_{f}}}{{n}_{m} ! {n}_{f} !} {x}_{{α}_{m}}^{{n}_{m}} {x}_{{α}_{f}}^{{n}_{f}} + {\sum }_{{n}_{m} , {n}_{f} = 1}^{∞} \frac{{c}_{{α}_{m}}^{{n}_{m}} {c}_{{α}_{a}}^{{n}_{a}}}{{n}_{m} ! {n}_{a} !} {x}_{{α}_{m}}^{{n}_{m}} {x}_{{α}_{a}}^{{n}_{a}} + {\sum }_{{n}_{f} , {n}_{a} = 1}^{∞} \frac{{c}_{{α}_{f}}^{{n}_{m}} {c}_{{α}_{a}}^{{n}_{a}}}{{n}_{f} ! {n}_{a} !} {x}_{{α}_{f}}^{{n}_{f}} {x}_{{α}_{a}}^{{n}_{a}} + ( {\sum }_{{n}_{m} , {n}_{f} , {n}_{a} = 0}^{∞} \frac{{c}_{{α}_{m}}^{{n}_{m}} {c}_{{α}_{f}}^{{n}_{f}} {c}_{{α}_{a}}^{{n}_{a}}}{{n}_{m} ! {n}_{f} ! {n}_{a} !} {x}_{{α}_{m}}^{{n}_{m}} {x}_{{α}_{f}}^{{n}_{f}} {x}_{{α}_{a}}^{{n}_{a}} ) \qquad [ Expression 6 ]$$

In expression (6), the second to fourth terms on the right side are each a term composed of a single feature amount, and represent a part of involvement to the characteristic Q that is related to factors other than the interaction. The fifth to seventh terms on the right side are each a term represented by the product of two types of feature amounts, and each of these terms can be regarded as a term indicating the contribution of the interaction between two types of element materials. Further, the eighth term on the right side is a term represented by the product of the three types of feature amounts, and can be regarded as a term indicating the contribution of the interaction by the three types of element materials.

Since it is difficult to further calculate the term represented by the infinite sum, it is preferable to convert each term of expression (6) into a form that does not include the infinite sum.

Specifically, the infinite series of each term is converted using a relational expression represented by the following expression (7).

$$\begin{matrix}{\sum }_{n = 1}^{∞} \frac{1}{n !} {c}^{n} {x}^{n} = {\sum }_{n = 0}^{∞} \frac{1}{n !} {c}^{n} {x}^{n} - 1 \\ = {e}^{cx} - 1\end{matrix} \qquad [ Expression 7 ]$$

From expression (7), relational expressions expressed by the following expression (8) and expression (9) can be further derived.

$$\begin{matrix}{\sum }_{{n}_{1} , {n}_{2} = 1}^{∞} \frac{1}{{n}_{1} ! {n}_{2} !} {c}_{1}^{{n}_{1}} {c}_{2}^{{n}_{2}} {x}_{1}^{{n}_{1}} {x}_{2}^{{n}_{2}} = ( {\sum }_{{n}_{1} = 1}^{∞} \frac{1}{{n}_{1} !} {c}_{1}^{{n}_{1}} {x}_{1}^{{n}_{1}} ) ( {\sum }_{{n}_{2} = 1}^{∞} \frac{1}{{n}_{2} !} {c}_{2}^{{n}_{2}} {x}_{2}^{{n}_{2}} ) = ( {e}^{{c}_{1} {x}_{1}} - 1 ) ( {e}^{{c}_{2} {x}_{2}} - 1 ) & [ Expression 8 ] \\ {\sum }_{{n}_{1} , {n}_{2} , {n}_{3} = 1}^{∞} \frac{1}{{n}_{1} ! {n}_{2} ! {n}_{3} !} {c}_{1}^{{n}_{1}} {c}_{2}^{{n}_{2}} {c}_{3}^{{n}_{3}} {x}_{1}^{{n}_{1}} {x}_{2}^{{n}_{2}} {x}_{3}^{{n}_{3}} = ( {\sum }_{{n}_{1} = 1}^{∞} \frac{1}{{n}_{1} !} {c}_{1}^{{n}_{1}} {x}_{1}^{{n}_{1}} ) ( {\sum }_{{n}_{2} = 1}^{∞} \frac{1}{{n}_{2} !} {c}_{2}^{{n}_{2}} {x}_{2}^{{n}_{2}} ) ( {\sum }_{{n}_{3} = 1}^{∞} \frac{1}{{n}_{3} !} {c}_{3}^{{n}_{3}} {x}_{3}^{{n}_{3}} ) = ( {e}^{{c}_{1} {x}_{1}} - 1 ) ( {e}^{{c}_{2} {x}_{2}} - 1 ) ( {e}^{{c}_{3} {x}_{3}} - 1 ) & [ Expression 9 ]\end{matrix}$$

Expression (10) can be derived by converting expression (6) using expressions (7) to (9).

$${y}_{α} = A ( 1 + ( {e}^{{c}_{{α}_{m}} {x}_{{α}_{m}}} - 1 ) + ( {e}^{{c}_{{α}_{f}} {x}_{{α}_{f}}} - 1 ) + ( {e}^{{c}_{{α}_{a}} {x}_{{α}_{a}}} - 1 ) + ( {e}_{{c}_{{α}_{m}} {x}_{{α}_{m}}} - 1 ) ( {e}^{{c}_{{α}_{f}} {x}_{{α}_{f}}} - 1 ) + ( {e}^{{c}_{{α}_{m}} {x}_{{α}_{m}}} - 1 ) ( {e}^{{c}_{{α}_{a}} {x}_{{α}_{a}}} - 1 ) + ( {e}^{{c}_{{α}_{f}} {x}_{{α}_{f}}} - 1 ) ( {e}^{{c}_{{α}_{a}} {x}_{{α}_{a}}} - 1 ) + ( {e}^{{c}_{{α}_{m}} {x}_{{α}_{m}}} - 1 ) ( {e}^{{c}_{{α}_{f}} {x}_{{α}_{f}}} - 1 ) ( {e}^{{c}_{{α}_{a}} {x}_{{α}_{a}}} - 1 ) ) \qquad [ Expression 10 ]$$

Note that in expression (10), similarly to expression (6), the second to fourth terms on the right side are are each a term composed of a single feature amount, and represent a part of involvement to the characteristic Q that is related to factors other than the interaction. The fifth to seventh terms on the right side are each a term represented by the product of two types of feature amounts, and each of these terms can be regarded as a term indicating the contribution of the interaction between two types of element materials. Further, the eighth term on the right side is a term represented by the product of the three types of feature amounts, and can be regarded as a term indicating the contribution of the interaction by the three types of element materials.

As in the second to fourth terms on the right side of expression (6), the linear term cᵢxᵢ is also included in the second to fourth terms on the right side of expression (10). Therefore, by separating the linear term from the second to fourth terms of the right side of expression (10) and transforming it into expression (11), this prediction expression can be separated into the linear term, the nonlinear term by the single explanatory variable, the nonlinear term indicating the contribution of the interaction by the two types of element materials, and the nonlinear term indicating the contribution of the interaction by the three types of element materials.

$${y}_{α} = A ( 1 + {c}_{{α}_{m}} {x}_{{α}_{m}} + {c}_{{α}_{f}} {x}_{{α}_{f}} + {c}_{{α}_{a}} {x}_{{α}_{a}} + ( {e}^{{c}_{{α}_{m}} {x}_{{α}_{m}}} - 1 ) + ( {e}^{{c}_{{α}_{f}} {x}_{{α}_{f}}} - 1 ) + ( {e}^{{c}_{{α}_{a}} {x}_{{α}_{a}}} - 1 ) - {c}_{{α}_{m}} {x}_{{α}_{m}} - {c}_{{α}_{f}} {x}_{{α}_{f}} - {c}_{{α}_{a}} {x}_{{α}_{a}} + ( {e}_{{c}_{{α}_{m}} {x}_{{α}_{m}}} - 1 ) ( {e}^{{c}_{{α}_{f}} {x}_{{α}_{f}}} - 1 ) + ( {e}^{{c}_{{α}_{m}} {x}_{{α}_{m}}} - 1 ) ( {e}^{{c}_{{α}_{a}} {x}_{{α}_{a}}} - 1 ) + ( {e}^{{c}_{{α}_{f}} {x}_{{α}_{f}}} - 1 ) ( {e}^{{c}_{{α}_{a}} {x}_{{α}_{a}}} - 1 ) + ( {e}^{{c}_{{α}_{m}} {x}_{{α}_{m}}} - 1 ) ( {e}^{{c}_{{α}_{f}} {x}_{{α}_{f}}} - 1 ) ( {e}^{{c}_{{α}_{a}} {x}_{{α}_{a}}} - 1 ) ) \qquad [ Expression 11 ]$$

In expression (11), the second to fourth terms on the right side correspond to linear terms, the fifth to tenth terms on the right side correspond to nonlinear terms by a single explanatory variable, the eleventh to thirteenth terms on the right side correspond to nonlinear terms indicating contributions of interaction by two types of element materials, and the fourteenth term on the right side corresponds to a nonlinear term indicating contributions of interaction by three types of element materials. Therefore, the terms on the right side of expression (11) are separated into expression (12) to expression (15).

$$\begin{matrix}{{y}^{⌣}}_{i} = {c}_{{α}_{m}} {x}_{{α}_{m}} + {c}_{{α}_{f}} {x}_{{α}_{f}} + {c}_{{α}_{a}} {x}_{{α}_{a}} & [ Expression 12 ] \\ {{y}^{⌣}}_{n 1} = ( {e}^{{c}_{{α}_{m}} {x}_{{α}_{m}}} - 1 ) + ( {e}^{{c}_{{α}_{f}} {x}_{{α}_{f}}} - 1 ) + ( {e}^{{c}_{{α}_{a}} {x}_{{α}_{a}}} - 1 ) - {c}_{{α}_{m}} {x}_{{α}_{m}} - {c}_{{α}_{f}} {x}_{{α}_{f}} - {c}_{{α}_{a}} {x}_{{α}_{a}} & [ Expression 13 ] \\ {{y}^{⌣}}_{n 2} = ( {e}^{{c}_{{α}_{m}} {x}_{{α}_{m}}} - 1 ) ( {e}^{{c}_{{α}_{f}} {x}_{{α}_{f}}} - 1 ) + ( {e}^{{c}_{{α}_{m}} {x}_{{α}_{m}}} - 1 ) ( {e}^{{c}_{{α}_{a}} {x}_{{α}_{a}}} - 1 ) + ( {e}^{{c}_{{α}_{f}} {x}_{{α}_{f}}} - 1 ) ( {e}^{{c}_{{α}_{a}} {x}_{{α}_{a}}} - 1 ) & [ Expression 14 ] \\ {{y}^{⌣}}_{n 3} = ( {e}^{{c}_{{α}_{m}} {x}_{{α}_{m}}} - 1 ) ( {e}^{{c}_{{α}_{f}} {x}_{{α}_{f}}} - 1 ) ( {e}^{{c}_{{α}_{a}} {x}_{{α}_{a}}} - 1 ) & [ Expression 15 ]\end{matrix}$$

Expression (12) represents the magnitude of the linear term, expression (13) represents the magnitude of the nonlinear term other than the interaction, expression (14) represents the magnitude of the nonlinear term indicating the interaction by the two types of element materials, and expression (15) represents the magnitude of the nonlinear term indicating the interaction by the three types of element materials. The first term on the right side of the expression (14) represents the magnitude of the nonlinear term indicating the interaction between the matrix resin m and the filler f as the element materials, the second term on the right side represents the magnitude of the nonlinear term indicating the interaction between the matrix resin m and the additive a as the element materials, and the first term on the right side represents the magnitude of the nonlinear term indicating the interaction between the filler f and the additive a as the element materials.

Note that the values of expression (13) to expression (15) (or the values of the terms included in the right sides of the respective expressions) may be negative values. At this time, it is shown that the interaction represented by the above expression or the above term is related in a direction of lowering the characteristics of the composite material α. Therefore, in the following expressions, the absolute values of these values are used for indicating the sum of the influence of the interaction.

At this time, the magnitude of the entire nonlinear term can be expressed by the following expression (16).

$${{y}^{⌣}}_{n} = {{y}^{⌣}}_{n 1} + {{y}^{⌣}}_{n 2} + {{y}^{⌣}}_{n 3} \qquad [ Expression 16 ]$$

In addition, the magnitude of the entire nonlinear term indicating the interaction can be expressed by the following expression (17).

$${{y}^{⌣}}_{i} = {{y}^{⌣}}_{n 2} + {{y}^{⌣}}_{n 3} \qquad [ Expression 17 ]$$

By converting expression (6) in this way, it is possible to convert the output values indicating the contributions of the interactions (the magnitudes of the nonlinear terms indicated by expression (14) and expression (15) into a format that can be output separately from the output values indicating the other contributions.

In step S240, the degree of involvement of the specified interaction to the characteristics of the composite material is evaluated using the values calculated by these expressions.

For example, among the characteristic values predicted by expression (2), a degree Rn accounts for nonlinearity can be expressed by the following expression (18).

$${R}_{n} = \frac{{{y}^{⌣}}_{n}}{\vert {{y}^{⌣}}_{n} \vert + \vert {{y}^{⌣}}_{i} \vert} \qquad [ Expression 18 ]$$

Further, the ratio Ri of the interaction term in the nonlinear term can be expressed by the following expression (19).

$${R}_{i} = \frac{{{y}^{⌣}}_{i}}{\vert {{y}^{⌣}}_{n 1} \vert + \vert {{y}^{⌣}}_{i} \vert} \qquad [ Expression 19 ]$$

A ratio of the degree of involvement of the interaction by the two (or three) types of element materials to the characteristic relative to the degree of involvement of the interaction term to the characteristic can be expressed by the following expression (20).

$${{R}^{⌣}}_{ib} = \frac{{{y}^{⌣}}_{nb}}{\vert {{y}^{⌣}}_{n 2} \vert + \vert {{y}^{⌣}}_{n 3} \vert} \qquad [ Expression 20 ]$$

Note that in expression (20), b is the number of element materials involved in the interaction whose ratio is to be obtained (in the present embodiment, b=2 or 3).

The ratio of the degree of involvement of a particular interaction (for example, the ratio of the degree of involvement of interaction between the matrix resin m and the filler f, between the matrix resin m and the additive a. or between the filler f and the additive a relative to the degree of involvement in the characteristic) can be expressed by the following expression (21).

$${{R}^{⌣}}_{2 cd} = \frac{( {e}^{{c}_{c} {x}_{c}} - 1 ) ( {e}^{{c}_{d} {x}_{d}} - 1 )}{\begin{matrix}\vert ( {e}^{{c}_{{α}_{m}} {x}_{{α}_{m}}} - 1 ) ( {e}^{{c}_{{α}_{f}} {x}_{{α}_{f}}} - 1 ) \vert + \\ \vert ( {e}^{{c}_{{α}_{m}} {x}_{{α}_{m}}} - 1 ) ( {e}^{{c}_{{α}_{a}} {x}_{{α}_{a}}} - 1 ) \vert + \\ \vert ( {e}^{{c}_{{α}_{f}} {x}_{{α}_{f}}} - 1 ) ( {e}^{{c}_{{α}_{a}} {x}_{{α}_{a}}} - 1 ) \vert\end{matrix}} \qquad [ Expression 21 ]$$

Note that in expression (21), c and d are m, f, or a, respectively. However, e and d indicate different element materials.

Furthermore, in expression (21), the denominator can be set to yₙ expressed by expression (16), y_{I} expressed by expression (17), or the like. At this time, it is possible to obtain the ratios of the degree of involvement of the interaction by the particular two types of element materials to the characteristics relative to the degree to which each of the nonlinearity occupies and relative to the degree of involvement of the entire interaction in the characteristics.

Expression (18) to (21) represent a ratio of a magnitude of a particular interaction (for example, the number of element materials constituting the interaction) relative to the output value of the entire interaction group including a plurality of interactions. In addition, when an explanatory variable indicating a element material is input to equation (10), the degree of involvement of the interaction selected in step S120 to the characteristics of the composite material can be evaluated by using the values obtained from expressions (18) to (21).

For example, the degree of involvement of the entire interaction can be evaluated based on the magnitude of the value of expression (19). In addition, the ratio of the degree of involvement of the interaction between two types of element materials or the interaction between three types of element materials to the characteristics can be evaluated based on the magnitude of the value of expression (20). The ratio of the degree of involvement of the interaction between the matrix resin m and the filler f to the characteristics of the composite material α can be evaluated based on the magnitude of the value of expression (21).

In addition, the degree of involvement of the entire interaction (whether the interaction has a positive influence or a negative influence, and the magnitude thereof) may be evaluated based on the magnitude of the value of expression (18). From expression (18), it can be calculated whether the interaction has the effect of strengthening the characteristic or weakening the characteristic, and the degree of the effect. Note that when the value of expression (18) or expression (19) is not so large, it may be determined that the degree of involvement of the interaction in the characteristics of the composite material is not so large, and the evaluation of the interaction may be aborted. Thus, the values of expression (18) and expression (19) can be used to determine whether interaction needs to be considered.

Note that although the composite material α composed of the three components of the matrix resin m, the filler f, and the additive a is described in the present embodiment, a similar expression transforming from expression (2) is possible for a composite material including three or more components. When the number of components is k and interactions by two element materials to interaction by m element materials are considered, expression (12), expression (16), expression (17), expression (20), and expression (21) can be expressed by the following expression (22), expression (23), expression (24), expression (26), and expression (27), respectively.

$${{y}^{⌣}}_{l} = {\sum }_{i = 1}^{m} {c}_{i} {x}_{i} \qquad [ Expression 22 ]$$

Note that in expression (22), m is the number of element materials.

$$\begin{matrix}{{y}^{⌣}}_{n} = {\sum }_{k = 1}^{m} {{y}^{⌣}}_{nk} & [ Expression 23 ] \\ {{y}^{⌣}}_{i} = {\sum }_{k = 2}^{m} {{y}^{⌣}}_{nk} & [ Expression 24 ]\end{matrix}$$

Note that the right sides of expression (23) and expression (24) include the sum of expression (25) as a term.

$${{y}^{⌣}}_{nk} = {\sum }_{{l}_{1} < {i}_{2} < ⋯ < {i}_{k}} {\prod }_{α = 1}^{k} ( {e}^{{c}_{{i}_{α}} {x}_{{i}_{α}}} - 1 ) - {δ}_{k 1} {{y}^{⌣}}_{l} \qquad [ Expresion 25 ]$$

Note that the sum expressed in expression (25) is a sum for a combination obtained by arranging the k natural numbers I_{α} such that I_{α} on the left is closer to I_{α} on the right. In addition, δₖ₁ is a Kronecker delta, and is a value that is 1 when k=1 and is 0 otherwise.

$$\begin{matrix}{{R}^{⌣}}_{ib} = \frac{{{y}^{⌣}}_{nb}}{{\sum }_{i = 2}^{m} \vert {{y}^{⌣}}_{ni} \vert} & [ Expression 26 ] \\ {{R}^{⌣}}_{{bc}_{1} {c}_{2} … {c}_{b}} = \frac{{\prod }_{α = 1}^{b} ( {e}^{{c}_{α} {x}_{α}} - 1 )}{{\sum }_{{c}_{2} < ⋯ < {c}_{b}} \vert {\prod }_{α = 1}^{b} ( {e}^{{c}_{α} {x}_{α}} - 1 ) \vert} & [ Expression 27 ]\end{matrix}$$

Note that in expression (22) to expression (27), m is the number of element materials included in the composite material, and k is the number of element materials constituting each interaction. In addition, in expression (26) to expression (27), b is the number of element materials involved in the interaction of which the ratio is to be obtained, and expression (27) indicates the ratio at which the interaction by the element materials 1, 2, . . . , and b is involved in the characteristics among the b types of interactions

Using the values of expression (22) to expression (27), it is possible to evaluate the degree of involvement of the interaction selected in Step S120 to the characteristics of the composite material, in a similar manner to the case of using the values of expression (18) to expression (21).

### Method of Specifying Element Material

Another embodiment of the present invention based on the above-described new finding relates to a method of specifying an element material to be replaced among element materials of a composite material.

The influence of interaction evaluated in the manner described above can be used to specify an element material that, when replaced by another material, would either change characteristics greatly or would not change characteristics greatly.

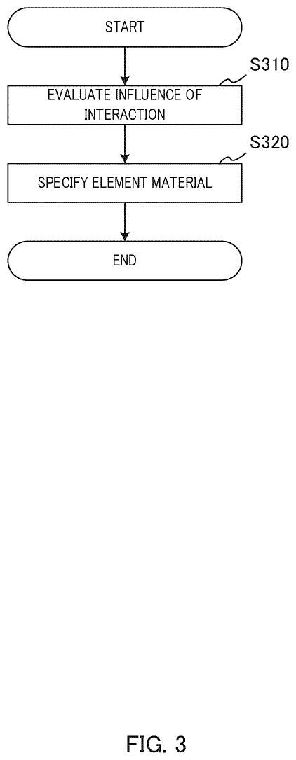

FIG. 3 is a flowchart illustrating a method of specifying an element material according to the present embodiment. The specifying method according to the present embodiment includes: a step of performing the above-described method of evaluating influence of interaction (step S310); and a step of specifying, based on the evaluated degree of involvement to the characteristics, an element material that greatly changes the characteristic or does not greatly change the characteristic when replaced with another material (step S320).

In the present embodiment, for a certain element material, the magnitude of the change in characteristics at the time of replacement is predicted based on the influence of the interaction involving the element material, and an element material is specified based on the predicted magnitude of the change. Since the prediction accuracy of the magnitude of the change in the characteristic at the time of replacement can be increased by considering the interaction, an element material that greatly changes the characteristic, an element material that does not greatly change the characteristic, or the like can be more accurately specified.

The element material to be specified may be one type of element material or two or more types of element materials.

In step S310, the above-described method of evaluating influence of interaction is performed. When it is preliminarily determined in Step S220 that the degree of the magnitude of the interaction is small, or when it is determined that the proportion of the nonlinear term in expression (18) or expression (19) or the entire interaction involved in the characteristics is small, the influence of the interaction on the characteristics of the composite material is considered to be small, and the present embodiment may be ended, and another method may be used to specify the element material. However, also in these cases, for example, by using another method and the method according to the present embodiment in combination, it is possible to specify the element material with higher accuracy in consideration of the influence of the interaction.

In a step S320, an element material is specified based on the evaluation result.

For example, when it is desired to specify an element material that largely changes characteristics when the element material is replaced by another material, an element material constituting the interaction, in which the value of the expression (27) (expression (21)) is large, may be specified. Further, when it is desired to specify an element material which does not largely change the characteristics when it is replaced by another material, an element material constituting the interaction, in which the value of the expression (27) (expression (21)) is small, may be specified. Note that the respective element materials appear in a plurality of terms on the right side of expression (11) (the right sides of expression (13) to expression (15)). Therefore, the above specifying may be performed based on the sum of all the terms including the element materials.

In addition, for example, when the degree to which the characteristics are desired to be changed from those of the original composite material is determined in advance, it is also possible to specify an element material such that the value of expression (27) (expression (21)), the sum of all terms including the element material, or the hike becomes the same degree as the degree to which the characteristics are desired to be changed.

The method according to the present embodiment can be used for determining which element material should be replaced with an alternative material according to the magnitude of the change in characteristics in considering replacement with the alternative material.

### Method of Searching for Alternative Material

In addition, still another embodiment of the present invention based on the above-described new finding relates to a method of searching for an alternative material that replaces an element material included in a composite material.

After the element material to be replaced with the alternative material is specified among the element materials included in the composite material by the above-described method of searching for an alternative material or other methods, it is necessary to consider which alternative material should be used to replace the element material. However, as described above, the characteristics of composite materials are influenced by interaction, and it is difficult to predict, from alternative materials, which alternative material will cause how much interaction. Therefore, there remains a problem of how to search for an alternative material that can exhibit desired characteristics.

For this problem, it is possible to search for an alternative material according to the degree of change in characteristics by unsupervised learning based on the feature amount.

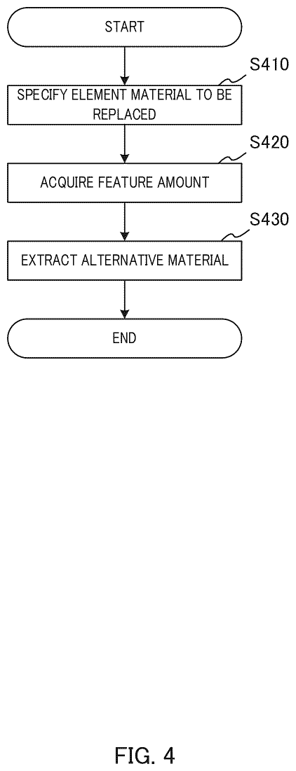

FIG. 4 is a flowchart illustrating a method of searching for an alternative material according to the present embodiment. The search method according to the present embodiment includes a step (step S410) of specifying an element material to be replaced among a plurality of types of element materials, a step (step S420) of acquiring a feature amount of the element material to be replaced, and a step (step S430) of extracting an alternative material from alternative material candidates by unsupervised learning based on the acquired feature amount.

In step S410, the element material to be replaced is specified. In this step, an element material that significantly changes the characteristics of does not significantly change the characteristics when replaced may be specified by the above-described method, or an element material (for example, a material that greatly affects the environment and is desired to be replaced) may be specified by another method.

In a step S420, the feature amount of the element material to be replaced specified in the previous step is acquired. Any type of feature amount may be used as long as it can be used to extract an alternative material by unsupervised learning in the next step.

For example, when the chemical structures of element materials to be replaced are known, multidimensional descriptors may be calculated using software such as alvaDese, RDKit, Mordred, XenonPy, or HSPiP, based on information in which the chemical structures are represented as character strings in SMILES, SMARTS, InChI, SELFIES, or the like.

Alternatively, multidimensional data may be generated by known image processing from image data (e.g., photographic data of the material) or moving image data of the element material to be replaced, and the multidimensional data may be used as the feature amount.

In addition, multidimensional data obtained by analyzing other components of an infrared absorption spectrum or the like may be used as the feature amount.

Furthermore, information obtained by five senses, such as a tactile sensation, a taste, and an aroma, may be used as the feature amount.

In addition, the following information that can be obtained from element materials can be used as feature amounts without limitation: electromagnetic spectra such as X-rays, ultraviolet light, visible light, near-infrared light, far-infrared light, and terahertz waves, physical property measurement data such as stress strain data by a tensile compression test, data measured by DSC or dynamic viscoelasticity, thermophysical property data such as melting point (Tm) or glass transition temperature (Tg), measurement values of GPC or HPLC, time changes of refractive index, transmittance, or absorbance, measurement values of NMR, density, particle size distribution, zeta potential. fluorescence or phosphorescence emission, thermal conductivity, electrical conductivity, measurement values of acoustic waves (reflection data obtained when acoustic waves are irradiated), and the like. Note that these pieces of information may be predicted values calculated by calculation.

In addition, when searching for an alternative material for a composite material having a more complicated composition, these pieces of information may be used in combination in order to realize a high-dimensional modality.

In a step S430, an alternative material capable of obtaining a new composite material having desired characteristics is extracted from candidates for the alternative material

Candidates for the alternative material are, for example, a material group including a plurality of materials such as known materials that can be used for the same purposes (base material resin, filler, various additives, and the like) as the element materials to be replaced, and materials listed in samples from manufacturers as materials to be used for the aforementioned purposes. It is preferable that the candidates for the alternative material are compiled into a database.

First, a feature amount is acquired for each of these candidates for the alternative material. The feature amount of the alternative material candidate acquired at this time is the same type of feature amount as the feature amount acquired from the element material to be replaced in the previous step. In other words, in the previous step, a feature amount that can be acquired from the candidates of the alternative material is selected, and the selected feature amount of the element material to be replaced is acquired. For example, when a candidate for the alternative material is a compound whose chemical structure is known, feature amounts derived from the chemical structures of the element material to be replaced and the candidate for the alternative material may be acquired in the previous step and the present step. Alternatively, when a candidate for the alternative material is a compound that causes even a slight difference in appearance such as the state of powder or the color tone of a solution or a dispersion liquid. feature amounts may be acquired from the image data of the element material to be replaced and the candidate for the alternative material in the previous step and the present step.

Next, based on the feature amounts of the element material to be replaced and the candidates for the alternative material, an alternative material that can provide a new composite material having desired characteristics is extracted from the candidates for the alternative material by unsupervised learning.

In the unsupervised learning, extraction can be performed, for example, based on the similarity between the feature amount of an element material to be replaced and the feature amount of each of candidates for a alternative material. Specifically, the extraction is performed based on a distance between a coordinate value indicating the element material and coordinate values indicating the alternative material candidates in a feature amount space in which the element material and the alternative material candidates are mapped based on the feature amounts. At this time, for example, when it is desired not to greatly change the characteristics of the original composite material, an alternative material having a high similarity to the feature amount of the element material to be replaced may be extracted, and when it is desired to change the characteristics of the original composite material, an alternative material having a low similarity to the feature amount of the element material to be replaced may be extracted. In addition, when a multidimensional feature amount is used, the unsupervised learning may be performed based on a vector in which a direction between a coordinate value indicating an element material and a coordinate value indicating a candidate for an alternative material is considered. In addition, when a multidimensional feature amount is used, different weights may be given to feature amounts based on a relationship with a desired characteristic.

As the mapping method, any one of known methods such as ISOMAP, PCA, kernel-PCA, sparse-PCA, sparse-kernel-PCA, LLE, t-SNE, spectral embedding, auto encoder, multidimensional scaling, and laplacian eigen map can be used.

As the distance and the similarity, it is possible to use Euclidean distance, Mahalanobis distance, Manhattan distance, Chebyshev distance, Minkowski distance, cosine similarity, Pearson product-moment correlation coefficient, and the like.

In this way, an alternative material with which a new composite material having desired characteristics can be obtained can be extracted from the candidates of the alternative material.

### EXAMPLE

For a composite material composed of three components (element materials) of a matrix resin, a filler and an additive, interactions between the element materials were evaluated, an element material to be replaced is specified based on the evaluated degrees of the interactions, and an alternative material was extracted so that a bending flexural modulus set as a characteristic is not largely changed.

First, nine types of materials were prepared for each of the element materials (27 types in total). One type of matrix resin, one type of filler, and one type of additive, each of which were selected from these materials, were charged into a twin-screw kneader (manufactured by Xplore, MC15) at respective predetermined addition rates, and were kneaded at 230° C. and a rotation speed of 130 rpm, to produce a composite material. Thirty-eight types of composite materials with different types and addition rates of element materials were produced, and the flexural modulus of each composite material was measured.

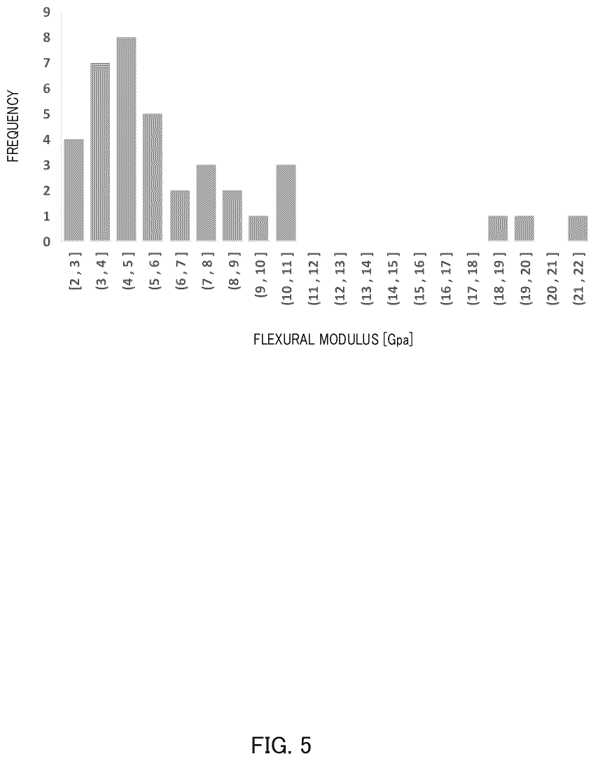

FIG. 5 is a histogram of the flexural modulus of the 38 types of composite materials, in which the horizontal axis represents the flexural modulus (GPa) and the vertical axis represents the frequency. On the basis of this histogram, a total of six pieces of data, i.e., three pieces of data having a flexural modulus of 15 GPa or more and three pieces of data having a flexural modulus of 15 GPa or less, were selected as test data.

Using the remaining 32 pieces of data as teacher data, two prediction expressions, expression (1) and expression (2), were created using the Partial Least Square (PLS) method (step S110, step S210). Note that the objective variable of expression (1) was the flexural modulus, and the objective variable of expression (2) was the logarithmic value of the flexural modulus. In addition, explanatory variables of expression (1) and expression (2) were all addition rates of the respective element materials. The number of latent variables was set to the smallest number of latent variables such that the root mean square residual (RMSE) was small when providing a prediction expression of PLS composed of latent variables from 1 variable to 10 variables, performing the Leave One Out cross validation (LOOCV), and calculating the RMSE. The number of latent variables set was four in both expression (1) and expression (2).

In this test, the degree of involvement to the magnitude of the flexural modulus, which is a characteristic, is evaluated for the interaction between two types of element materials or the interaction between three types of element materials (step S120).

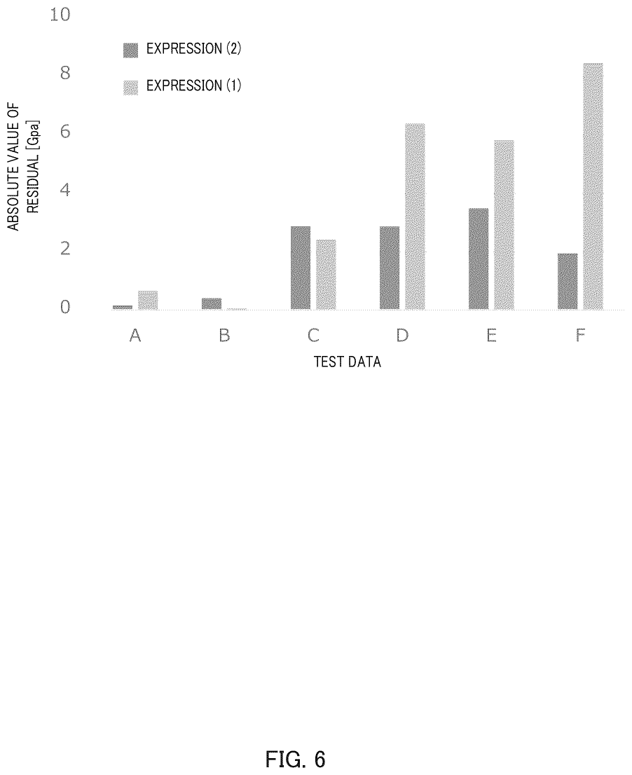

For the six pieces of test data, the absolute values of the residuals between the predicted values of the flexural modulus (or the logarithmic value thereof) calculated from expression (1) and expression (2), respectively, and the actually measured flexural modulus (or the logarithmic value thereof) were obtained (step S220). FIG. 6 is a graph illustrating the absolute values of the residuals according to expression (1) and expression (2) for each of the six pieces of test data. Note that the test data A to test data F are data having the actual measurement values of the flexural modulus are the following values.

| | | |
|---|---|---|
| Test data A | 3.3 | GPa |
| Test data B | 5.8 | GPa |
| Test data C | 10.5 | GPa |
| Test data D | 17.9 | GPa |
| Test data E | 19.2 | GPa |
| Test data F | 21.2 | GPa |

It can be seen from FIG. 6 that the absolute values of the residuals according to expression (2) are smaller than the absolute values of the residuals according to expression (1) particularly in the test data D to the test data F, having an flexural modulus equal to or more than 15 GPa. That is, in these three pieces of data, it can be said that the degree of influence of the nonlinear term on the predicted value is large and the contribution of the interaction in the measured value is large. Therefore, in the following study, the test data D was used to evaluate the degree of involvement of the interaction to the characteristics (flexural modulus) of the composite material.

Expression (2) set above was converted into the format of expression (11) and separated into expression (12) (the magnitude of the linear term), expression (13) (the magnitude of the nonlinear term other than the interaction), expression (14) (the magnitude of the nonlinear term indicating the interaction between two types of element materials), and expression (15) (the magnitude of the nonlinear term indicating the interaction between three types of element materials) (step S230). Then, Rn (the degree to which the nonlinearity occupies the characteristic value predicted by the expression (2)) was obtained by the expression (18), Ri (the degree to which the entire interaction involves in the characteristic) was obtained by the expression (19), Rib (the ratio of the degree to which the interaction between the two (or three) types of element materials involves in the characteristic) was obtained by the expression (20), and R_{mf}, Rₘₐ and R_{fa} were obtained by the expression (21).

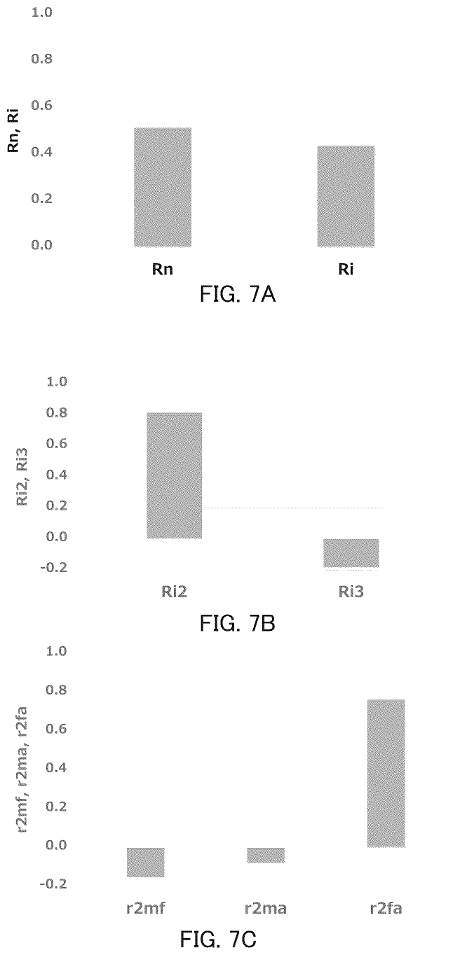

FIG. 7A is a graph illustrating Rn obtained from test data D according to expression (18) and Ri obtained from test data D according to expression (19), FIG. 7B is a graph illustrating Rᵢ₂ and Rᵢ₃ obtained from test data D according to expression (20), and FIG. 7C is a graph illustrating R_{2mf}, R₂ₘₐ and R_{2fa} obtained from test data D according to expression (21).

The ratio Rn of the nonlinear term in FIG. 7A was 0.497. From this result, it can be seen that the nonlinear term contributes to increase the flexural modulus m the test data D, and the contribution thereof accounts for about 50% of the total of the linear term and the nonlinear term. The magnitude of the actual nonlinear term in test data D is calculated to be 5.63 GPa, which is a non-negligible magnitude with respect to the flexural modulus of test data D (17.9 GPa).

The ratio Ri of the interaction term illustrated in FIG. 7A was 0.440. From the results, it is found that in the test data D, the interaction contributes to increase the flexural modulus, and the contribution accounts for about 44% of the entire nonlinear term. The magnitude of the actual interaction term in the test data D is calculated to be 2.46 GPa, which is also not a minute value.

The ratio Rᵢ₂ of the interaction by the two types of element materials in FIG. 7B was 0.82, and the ratio Rᵢ₃ of the interaction by the three types of element materials was 0.18. From this result, it can be seen that the interaction by the two types of element materials contributes to increase the flexural modulus and the contribution thereof accounts for about 80% of the contribution of the entire interaction, and that the interaction by the three types of element materials contributes to decrease the flexural modulus and the contribution thereof accounts for about 20% of the contribution of the entire interactions.

The respective ratios of interactions by the two types of element materials in FIG. 7C were 0.16 for R_{2mf}, 0.08 for R₂ₘₐ and 0.76 for R_{2fa}. From the results, it is found that both of the interaction between the base material resin and the filler and the interaction between the base material resin and the additive contribute to decrease the flexural modulus, and the contributions thereof account for about 16% and about 8%, respectively, of the contribution of the entire interactions. It can also be seen that any interaction between the fillers and additives contributes to increasing the flexural modulus, which accounts for about 76% of the contribution of the entire interactions (step S240, these steps are also step S130 and Step 310).

From the results, it is found that the degree of change in flexural modulus (characteristics) is greater when the filler or additive is replaced with an alternative material than when the base material resin is replaced with an alternative material. From this result, it is possible to consider replacing the matrix resins so that the flexural modulus does not change as much as possible, but in the present example, an additive, which is more likely to change the flexural modulus, is replaced with an alternative material in order to verify the magnitude of change in characteristics due to replacement with the alternative materials (step S320, step S410).

Simplified molecular input line entry system (SMILES) data on compounds manufactured by Tokyo Chemical Industry Co., Ltd. (TCI), which were registered in the chemical molecular database Pubchem, were obtained, and 14180 compounds containing no metal atom and having a molecular weight of 100 or more and 900 or less were extracted therefrom. Using the molecular expression description calculation program alvaDesc, 3885 two-dimensional molecular descriptors were calculated for each of the extracted compounds. Thereafter, the 3885 two-dimensional molecular descriptors were introduced into a prediction expression for decomposition temperature prepared in advance to predict the decomposition temperature of each compound, and 1309 compounds having a probability of 80% or more of having a decomposition temperature of 240° C. or more so as to withstand the heating temperature (230° C.) by the kneader were selected as candidates for alternative materials for additives.

As the prediction expression of the decomposition temperature, the following was used: a prediction expression prepared by using 20 compounds for which measured values of decomposition temperatures and SMILES data of chemical structures are known, and by a Gaussian process regression method using the RBF kernel with the decomposition temperature as the objective variable and two-dimensional molecular descriptor created by alvaDese as the explanatory variable. As the probability that the decomposition temperature became 240° C. or more, a value obtained by integrating the probability distribution obtained from the prediction expression in a region of 240° C. or more was used.

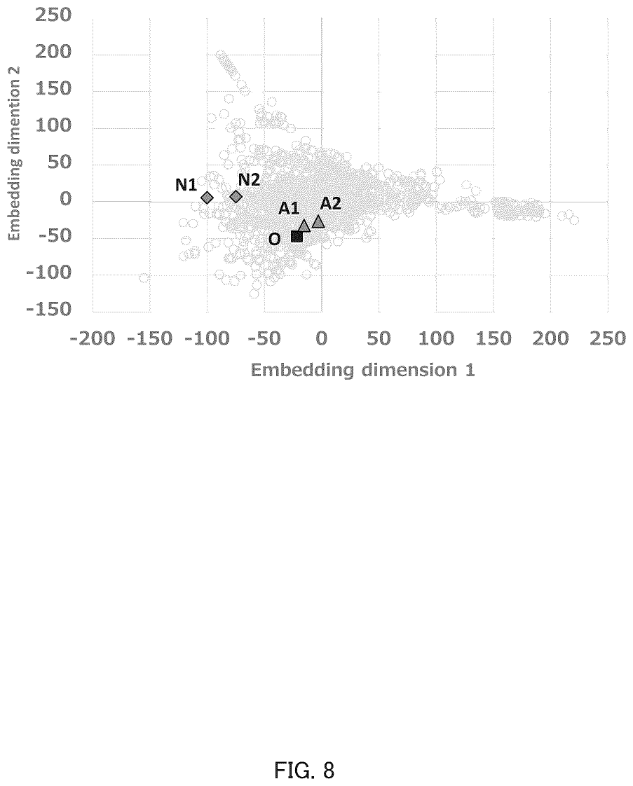

Next, principal component analysis was performed using the additives used in the test data D and the above-selected alternative material candidates (a total of 1310 compounds), and the obtained 400 principal components from the 1st principal component to the four hundredth principal component were used as feature amounts (step S420). ISOMAP was performed using the above 1310 compounds and the above feature amounts. FIG. 8 shows an execution result of the ISOMAP. The additive used in the test data D is indicated by “O” in FIG. 8. The compounds “A1” and “A2” close to the point “O” and the compounds “N1” and “N2” far from the point “O” in the distribution illustrated in FIG. 8 were extracted as alternative materials (step S430).

Composite materials were prepared under the same conditions as in Test D, except that each additive thereof was a compound “A1”, “A2”, “N1” or “N2”, and the flexural modulus was measured. The test data D and the additives of respective composite materials, the measured flexural modulus, and the difference Δ in flexural modulus between the test data D and each composite material are shown in Table 1.

TABLE 1

| Composite | | Flexural | |
|---|---|---|---|
| material | Additive | modulus (Gpa) | Δ (Gpa) |
| Test data D | O | 17.89 | 0.00 |
| No. 1 | A1 | 18.51 | 0.62 |
| No. 2 | A2 | 18.80 | 0.91 |
| No. 3 | N1 | 20.10 | 2.21 |
| No. 4 | N2 | 12.86 | 5.03 |

It is found from Table 1 that when the additive “A1” or “A2” having distance to the additive “O” is short in the test data D based on the feature amounts is used as the alternative material, the flexural modulus of the composite material after the replacement does not change much. Furthermore, it is found that when the additive “N1” or “N2” having distance to the additive “O” is long in the test data D based on the feature amounts is used as the alternative material, the flexural modulus of the composite material after the replacement is greatly changed. Therefore, it is understood that the additive “A1” or “A2” may be used as an alternative material when it is desired to obtain a new composite material in which the characteristic (flexural modulus) of the test data D is maintained as much as possible, and the additive “N1” or “N2” may be used as an alternative material when it is desired to obtain a new composite material in which the characteristic (flexural modulus) of the test data D is changed.

### Other Embodiments

Note that the above-described embodiment is merely an example of embodying the present invention, and the technical scope of the present invention should not be interpreted in a limited manner by the above-described embodiment. The present invention can be implemented in various forms without departing from the gist or main features thereof.

For example, although the flexural modulus of the composite material is used as a characteristic for which the degree of change is considered in each of the above-described embodiments, other various mechanical characteristics, thermal characteristics, electrical characteristics, and the like (e.g., strength such as tensile strength, compressive strength, and shear strength, hardness, elongation at break, impact strength, abrasion resistance, flame retardancy, heat resistance, light resistance, weather resistance, acid resistance, alkali resistance, solvent resistance, and color tone) may be used as characteristics for which the degree of change is considered. Further, the degree of change in not only one type of characteristic but also a plurality of types of characteristics may be examined.

In addition, in the above-described method of evaluating influence of interaction, the addition amount of each component is used as the feature amount, but the feature amounts listed as the feature amounts of the element materials in the method of searching for an alternative material or a combination thereof may be used as the feature amount in the method of evaluating influence of interaction.

Furthermore, although the composite material containing the three types of element materials, namely the base material resin, the filler, and the additive, is given as an example in each of the above-described embodiments, the types and the number of the element materials are not limited thereto, and the type of the composite material is also not particularly limited. For example, an adhesive or an ink material, a fragrance, food or a pharmaceutical product, a biomaterial, a sensor, or the like may be used as the composite material.

For example, in the case of an adhesive, when two different types of molded articles are bonded, there are bonding including interaction at each interface and/or in the vicinity of the interface, and in the case where the molded article is a polymer, there is entanglement between polymers in the vicinity of the interface between the molded article and the adhesive, and the strength of the bonding, the entanglement, and the adhesiveness change nonlinearly depending on the components and the formulation contained in the composite material, and therefore, the technique of the present invention can be applied.

Furthermore, for example, in cell culture or the like of a biological material, the effect is nonlinear due to the influence of a medium, pH, osmotic pressure, CO2, oxygen, environmental conditions such as temperature and the like, essential nutrients (amino acids, carbohydrates, vitamins, minerals), growth factors, hormones and the like, and therefore, the technique of the present invention can be applied.

In order to detect cancer cells or the like, there is a method in which light emitting sources are combined. As the light emitting source, for example, a composite material of a resin/a surfactant/luminescent particles may be used, and the method of the present invention can be applied because effects such as the amount of emitted light, durability, and binding properties with cells become nonlinear depending on the composite material, the preparation step conditions of the luminescent particles, and the components and blending of the composite material.

In addition, since the bio-adaptive material is a material which is adapted to a living body environment, positively utilizes interaction between the living body and the material, and exhibits a function, the method of the present invention can be applied.

The present application claims the benefit of Japanese Patent Application No. 2021-201146 filed on Dec. 10, 2021, and the disclosures in the specification, claims, abstract and drawings of the application are incorporated herein by reference.

### INDUSTRIAL APPLICABILITY

The present invention is useful for determination and search of alternative materials for composite materials.

1. A method of interaction, the method comprising:
selecting, for a composite material including a plurality of types of element materials, a type of interaction by two or more element materials of the plurality of types of element materials; and
evaluating a degree of involvement of the selected interaction to a characteristic possessed by the composite material.

2. The method of evaluating influence of interaction according to claim 1, wherein:
in the evaluating, an explanatory variable indicating an element material of the plurality of types of element materials is input to a prediction model that predicts the characteristic of the composite material;
the prediction model is capable of outputting an output value indicating a contribution of the interaction separately from an output value indicating another contribution; and
the degree of involvement of the selected interaction is evaluated based on the output value indicating the contribution of the interaction obtained from the prediction model.

3. The method of evaluating influence of interaction according to claim 2, wherein the prediction model is a prediction model that predicts the characteristic by a nonlinear prediction expression expressed by an exponential function.

4. The method of evaluating influence of interaction according to claim 3, wherein the output value indicating the contribution of the interaction is an output value from a term including two or more different feature amounts in an expression obtained by Taylor expansion of the nonlinear prediction expression.

5. The method of evaluating influence of interaction according to claim 3, wherein:
in the evaluating, a magnitude of a nonlinear term in the output value from the prediction model is determined; and
the degree of involvement of the selected interaction is evaluated as sufficiently large when the magnitude of the nonlinear term is sufficiently large.

6. The method of evaluating influence of interaction according to claim 2, wherein:
in the evaluation, a ratio of a magnitude of an output value of the selected interaction to a total output value of an interaction group composed of a plurality of interactions is calculated; and
the degree of involvement of the selected interaction is evaluated based on the calculated ratio.

7. A method of specifying an element material, comprising:
performing the method of evaluating influence of interaction according to claim 1; and
specifying, based on the degree of involvement obtained by the method of evaluating influence of interaction, the element material corresponding to a degree to which the characteristic changes when the element material is replaced.

8. A method of searching for an alternative material that replaces an element material of a composite material including a plurality of types of element materials, the element material being among the plurality of types of element materials, the method comprising;
specifying the element material to be replaced among the plurality of types of element materials by the method of specifying an element material according to claim 7;
acquiring a feature amount of the element material to be replaced; and
extracting an alternative material from a candidate for the alternative material based on the acquired feature amount.

9. The method of searching for an alternative material according to claim 8, wherein, in the extracting, the alternative material is extracted by unsupervised learning.

10. The method of searching for an alternative material according to claim 9, wherein, in the extracting, the alternative material is extracted by the unsupervised learning based on similarity between the feature amount of the element material acquired in the acquiring and a feature amount of the candidate for the alternative material.

11. The method of searching for an alternative material according to claim 9, wherein, in the extracting, the alternative material is extracted by the unsupervised learning based on at least one of a distance and/or a similarity between a coordinate value indicating the element material and a coordinate value indicating the candidate for the alternative material in a feature amount space in which the element material and the candidate for the alternative material are mapped based on the feature amount.

12. The method of searching for an alternative material according to claim 10, wherein the candidate for the alternative material is a compound whose chemical structure is known, and the feature amount of the element material is derived from the chemical structure, and the feature amount of the candidate for the alternative material is derived from a chemical structure of the candidate.

13. The method of searching for an alternative material according to claim 8, wherein the feature amount of the element material is a feature amount indicated as multidimensional data.

14. The method of searching for an alternative material according to claim 13, wherein the feature amount is a feature amount obtained from an image or a moving image.
